(19) United States
(12) Patent Application Publication
(10) Pub. No.: US 2019/0365785 A1
(43) Pub. Date: Dec. 5, 2019

(54) INHIBITION OF UNFOLDED PROTEIN RESPONSE FOR SUPPRESSING OR PREVENTING ALLERGIC REACTION TO FOOD

(71) Applicants: Children's Hospital Medical Center, Cincinnati, OH (US); University of Cincinnati, Cincinnati, OH (US)

(72) Inventors: Fred Finkelman, Cincinnati, OH (US); Marat Khodoun, Cincinnati, OH (US); Suzanne Morris, Mason, OH (US); Unni Krishna Sri Rama Lingeswa Samavedam, Cincinnati, OH (US); Simon Patrick Hogan, Witmore Lake, MI (US)

(21) Appl. No.: 16/476,608

(22) PCT Filed: Feb. 19, 2018
(86) PCT No.: PCT/US18/18618

§ 371 Date: Jul. 9, 2019

Related U.S. Application Data
(60) Provisional application No. 62/533,224, filed on Jul. 17, 2017.
(60) Provisional application No. 62/462,077, filed on Feb. 22, 2017.

(51) Int. Cl. A61K 31/575; A61K 31/155; A61K 31/216; A61K 39/35; A61P 37/08

(52) U.S. Cl. CPC A61K 31/575; A61K 31/155; A61P 37/08; A61K 39/35; A61K 31/216

(57) ABSTRACT

Provided are methods of preventing or suppressing an allergic reaction to food by administering an inhibitor of the unfolded protein response (UPR) and uses of a composition comprising an inhibitor of the UPR for treating or preventing a food allergy for manufacture of a medicament for treating or preventing a food allergy.

### CROSS-REFERENCE TO RELATED APPLICATIONS

This application claims the priority to and the benefit of U.S. Provisional Application Ser. No. 62/462,077, filed Feb. 22, 2017 and 62/533,224 filed Jul. 17, 2017, the contents of each are incorporated in their entirety for all purposes.

### BACKGROUND

Food allergy is a significant and growing healthcare problem. It is estimated that more than 15 million people in the United States alone—about 8% of children and about 4% of adults—suffer from allergies to one or more of the top eight major food allergens. 40% of those with food allergies are children. Furthermore, the incidence of food allergy has been rapidly increasing in the U.S. and other developed countries (Sicherer et al. 2010a; Sicherer et al. 2010b; Branum et al. 2009). Reactions to food allergens range from skin and gastrointestinal reactions to respiratory reactions, including anaphylaxis and potentially, death. In the United States, food allergy is responsible for 50,000 emergency room visits and about 150 deaths per year (Sicherer et al. 2010a; Sicherer et al. 2010b; Branum et al. 2009).

There is no approved therapy for this disorder, other than avoidance of foods that cause allergic symptoms, and administration of antihistamines, steroids, or epinephrine (depending on the severity of the reaction) once symptoms have developed. Avoidance can be very difficult. Although the eight major food allergens must be listed on packaged foods in the United States, cross-contamination can occur during manufacturing, resulting in the food product having a hidden allergen that does not appear on the label. In addition, food service businesses are not required to list food allergens, so an individual's safety depends on clear communication, on food service employees' knowledge of the allergen content of the food being served, and on the business' management practices. Even with the most stringent management practices, accidents, such as cross-contamination, can still occur when people with a food allergy eat outside of the home. Quality of life of food allergy sufferers can be significantly impacted by these factors. There is an unmet need for pharmacologic therapies to treat food allergy prior to an adverse reaction in this large, and largely neglected, patient population.

The instant disclosure addresses one or more of the aforementioned needs in the art.

### SUMMARY OF THE INVENTION

Provided are methods of preventing or suppressing an allergic reaction to food by administering an inhibitor of the unfolded protein response (UPR) and uses of a composition comprising an inhibitor of the UPR for treating or preventing a food allergy or for manufacture of a medicament for treating or preventing a food allergy.

### BRIEF DESCRIPTION OF THE FIGURES

FIG. 1 shows suppression by UPR inhibitors of pro-Th2 cytokine expression in a human intestinal epithelial cell line (CACO-2).

FIG. 2 shows reduced expression of UPR-related genes (A) and pro-Th2 cytokine genes (B) in CACO-2 cells treated with metformin.

FIG. 3 shows that lipase inhibition suppresses UPR-related and pro-Th2 cytokine gene expression in CACO-2 cells cultured with egg yolk plasma, but not in CACO-2 cells cultured with peanut extract.

FIG. 4 shows that the UPR inducer sodium palmitate and egg yolk plasma (EYP) increase pro-Th2 cytokine production by CACO2 cells.

FIG. 5A-5D show relative expression (versus GAPDH) of UPR-related and pro-Th2 cytokine genes in CACO-2 cells cultured for 6 hours (FIG. 5A, 5C) or 24 hours (FIG. 5B, 5D) with aqueous extracts of walnut (FIG. 5A-5B) or fish (FIG. 5C-5D).

FIG. 6A-6B show that the UPR inducer sodium palmitate induces a UPR-dependent increase in pro-Th2 cytokine expression by human intestinal organoids. Numbers to right of bars in FIG. 6B show percent inhibition for metformin/TUDCA.

FIG. 7 shows that IRE-1α is important for UPR induction of pro-Th2 cytokine expression. HPRT is hypoxanthine guanine phosphoribosyl transferase, a housekeeping gene used as an internal standard.

FIG. 8 shows that development of hypothermia in response to ingested allergen is IgE-dependent in food-allergic mice. BALB/c mice (4/group) were inoculated by oral gavage (o.g.) with medium chain triglycerides (MCT) for 3 days, then with medium chain triglycerides+egg white (MCT/EW) every other day until they developed hypothermia in response to o.g. inoculation. Mice were then injected i.p. with 500 μg of anti-IgE monoclonal antibody (mAb) (EM-95), 500 μg of anti-FcγRIIB/RIII mAb (2.4G2), both mAbs, or isotype control mAbs. One day later, mice were challenged o.g. with MCT/EW, and rectal temperatures were followed for the next 60 minutes. Asterisk indicates a statistically significant (p<0.05) difference between groups connected by the bracket.

FIG. 9 shows that Pro-Th2 cytokine antagonists have a lasting effect on development of food allergy. Panel A shows the treatment protocol. Briefly, BALB/c female mice, 4-6 mice per group, were inoculated o.g. with 100 μl of MCT on days 0 and 3, then inoculated o.g. with MCT/EW emulsion every other day for 3 weeks. One group was injected intraperitoneally (i.p.) with a cocktail of anti-TSLP/anti-IL-33R/anti-IL-25 mAbs 12 hours before each MCT/EW dose, while the other group was injected i.p. with isotype control mAbs. Rectal temperatures were determined for the hour after the last o.g. inoculation (Panel B, left) and mice were bled 4 hours after this inoculation. Treatment with anti-pro-Th2 cytokine mAbs and isotype control mAbs was then discontinued, but all mice were inoculated o.g. every other day for an additional 5 weeks with MCT/EW. Mice were again followed for decreases in rectal temperature for 1 hour after the last o.g. inoculation (Panel B, right). Mice were again bled 4 hours after this o.g. inoculation and total IgE, EW-specific IgG1, and mouse mast cell protease 1 (MMCP1) levels were evaluated by ELISA (Panel C). Asterisks indicate p<0.05, as compared to isotype control treated mice.

FIG. 10 shows that IL-25, IL-33, and TSLP are all required for development of food allergy in MCT/EW-inoculated mice. Panel A shows the treatment protocol. Briefly, BALB/c mice, 4-6/group, were fasted for 4 hours and left untreated or inoculated o.g. with 100 μl of MCT on day 0 and day 3. MCT-treated mice were then inoculated o.g. with MCT/EW emulsion every other day for three weeks. Mice were also injected i.p. 12 hours before each MCT/EW inoculation with anti-TSLP mAb, anti-IL-25 mAb, anti-IL-33R mAb, a cocktail of anti-TSLP/anti-IL-33/anti-IL-25 mAbs, or with isotype control mAbs 12 hours before each MCT/EW dose. Rectal temperatures were determined for the hour after the last o.g. inoculation (Panel B). Mice were bled 4 hours after this inoculation. IL-4, IL-13, and IFN-γ secretion were evaluated by in vivo cytokine capture assay (IVCCA); while serum levels of MMCP1, IgE, and IgG1 anti-EW were determined by ELISA (Panel C). Asterisks indicate a statistically significant (p<0.05) difference compared to isotype control treated mice and between groups connected by a bracket.

FIG. 11 shows that established food allergy is suppressed by an anti-pro-Th2 mAb cocktail. Panel A shows the treatment protocol. Briefly, BALB/c mice were fasted for 4 hours and sensitized with two oral doses of MCT on day 0 and day 3. Subsequently, mice were treated with MCT/EW emulsion every other day for four weeks. Mice that developed>4° C. maximum temperature drop were divided into 3 groups of 5 mice per group. All groups were inoculated o.g. with MCT/EW emulsion twice a week for 4 more weeks. The different groups were also injected i.p. with anti-TSLP mAb, with the cocktail of anti-TSLP/anti-IL-33R/anti-IL-25 mAbs, or with isotype control mAbs 12 hours before each MCT/EW inoculation. Decreases in rectal temperature were determined for the hour after the last MCT/EW inoculation (Panel B). Mice were bled 4 hours after the last o.g. inoculation for determination of serum MMCP1 levels (Panel C), as well as serum IgE levels and IgG1 anti-EW titers (Panel D). Asterisks indicate a statistically significant (p<0.05) difference compared to isotype control treated mice and between groups connected by a bracket.

FIG. 12 shows that combined pro-Th2 cytokine blockade is required for effective suppression of established food allergy. Panel A shows the treatment protocol. Briefly, BALB/c mice were fasted for 4 hours, then inoculated o.g. with 100 μl of MCT on day 0 and day 3. Mice were then kept unimmunized or were inoculated o.g. with MCT/EW emulsion twice a week for four weeks. Mice that developed significant shock (more than 4° C. maximum temperature drop) were divided into 5 groups of 5 mice/group. All groups were then inoculated o.g. with MCT/EW emulsion twice a week for an additional 3 weeks. Different groups of MCT/EW-immune mice were injected i.p. with the following mAb combinations 12 hours before each o.g. inoculation with MCT+EW: anti-TSLP+anti-IL-33R mAb; anti-TSLP+anti-IL-25 mAb, anti-IL-25+anti-IL-33R mAb, anti-TSLP+anti-IL-33R+anti-IL-25 mAb, or isotype control mAbs. Maximal decreases in rectal temperature were determined for the hour following the o.g. inoculation just prior to the initiation of mAb treatment (Panel B, day 0) and for the hour following the o.g. inoculations after 14 and 24 days of mAb treatment (Panel B). Mice were bled 4 hours after the day 24 o.g. inoculation to determine levels of IL-4 and IL-13 secretion, MMCP1 response, and serum IgE and IgG1 anti-EW levels (Panel C). Asterisks indicate a statistically significant (p<0.05) difference compared to isotype control treated mice and between groups connected by a bracket.

FIG. 13 shows that maintenance of increased lamina propria Th2 cell, mast cell (MC), and eosinophil numbers in food allergy is pro-Th2 cytokine-dependent. Panel A shows the treatment protocol. Briefly, BALB/c mice (4/group) were left untreated (naïve) or were inoculated o.g. with MCT for 3 days, followed by MCT/EW every 4 days for 5 weeks. Following this, mice that had developed a temperature drop of at least 2° C. following o.g. inoculation continued to receive o.g. MCT/EW every 4 days for an additional 5 weeks; half of these mice were injected i.p. with anti-TSLP/IL-25/IL-33 mAbs, half with isotype control mAbs, 4 hours before each o.g. inoculation. Following the last o.g. inoculation, lamina propria (LP) and mesenteric lymph node (MLN) single cell suspensions were prepared, stained for Th2 cell, ILC2, mast cell, basophil, eosinophil or dendritic cell markers and analyzed for number of each cell type by Coulter counting and flow cytometry (Panel B). Asterisks indicate a statistically significant (p<0.05) difference compared to isotype control treated mice and between groups connected by a bracket.

FIGS. 14A and 14B show suppression by UPR inhibitors of hypothermia (FIG. 14 A, Left Panel), MMCP1 response (FIG. 14 A, Right Panel), and small intestinal UPR-related and pro-Th2 cytokine gene expression (FIG. 14 B) in a mouse model of food allergy.

FIG. 15A-15E show response to treatment with UPR inhibitors in a mouse model of egg allergy. Control and treated mice were induced to develop egg allergy; naïve mice were not induced. Food allergy was established prior to treatment with UPR inhibitors. In FIG. 15E, data are pooled from 2 experiments; n=5 naïve mice, 13 control mice, 15 metformin-treated mice, and 11 TUDCA-treated mice.

FIG. 16A-16B show that oral metformin suppresses egg yolk plasma-induced pro-Th2 cytokine and UPR-related gene expression in skin (FIG. 16A) and lung (FIG. 16B) of mice with established egg allergy.

### DETAILED DESCRIPTION OF THE INVENTION

Unless defined otherwise, all technical and scientific terms used herein have the same meaning as commonly understood by one of ordinary skill in the art to which this invention is related.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Système International de Unites (SI) accepted form. Numeric ranges are inclusive of the numbers defining the range. The headings provided herein are not limitations of the various aspects or embodiments of the invention, which can be had by reference to the specification as a whole. Accordingly, the terms defined immediately below are more fully defined by reference to the specification in its entirety.

Wherever embodiments are described with the language “comprising,” otherwise analogous embodiments described in terms of “consisting of” and/or “consisting essentially of” are included.

The “unfolded protein response” or “UPR” is an endoplasmic reticulum stress response characterized by upregulation of UPR-related genes, including protein kinase RNA-like endoplasmic reticulum kinase (PERK), binding immunoglobulin protein (BiP), CCAAT/enhancer-binding protein homologous protein (CHOP), activating transcription factor 4 (ATF4), activating transcription factor 6 (ATF6), endoplasmic reticulum to nucleus signaling 1 (ERN1, which encodes inositol-requiring enzyme 1 (IRE1)), X-box binding protein 1 (XBP1), and XBP1 spliced protein (XBP1s). Certain food allergens promote food allergy by inducing an epithelial cell UPR, which in turn, causes these cells to express pro-Th2 cytokines, including IL-25, IL-33, and thymic stromal lymphopoietin (TSLP).

An “active agent” is an agent which itself has biological activity, or which is a precursor or prodrug that is converted in the body to an agent having biological activity. Active agents useful in the methods of the invention include UPR inhibitors.

An “inhibitor of UPR” or “UPR inhibitor” is an active agent that suppresses expression of at least one UPR-related gene, protein, or signaling pathway. Examples of UPR inhibitors include 4-(2-aminoethyl)benzenesulfonyl fluoride hydrochloride (AEBSF); 5-aminoimidazole-4-carboxamide ribonucleotide (AICAR); 4-phenylbutyrate (4-PBA); bile acids (e.g., UDCA and TUDCA); Binding immunoglobulin protein (BiP); ceapins (Gallagher et al. 2016); extendin-4; GSK2606414; IC87144; IRE1 inhibitors (Tomasio et al. 2013); metformin; rapamycin; salubrinal; SRT1720; STF-083010; toyocamycin; and vaticanol B (Tabata et al. 2007). The term “UPR inhibitor” includes phosphorylated forms and pharmaceutically acceptable salts of the disclosed compounds. UPR inhibitors also include nucleic acid or polypeptide inhibitors of UPR-related genes or gene expression products, for example, siRNA, miRNA, shRNA, dominant-negative polypeptides, inhibitory peptides, blocking antibodies, and oligonucleotide or polypeptide aptamers, the synthesis of which will be readily appreciated by one of ordinary skill in the art.

The terms “inhibit,” “block,” and “suppress” are used interchangeably and refer to any statistically significant decrease in biological activity, including full blocking of the activity.

By “subject” or “individual” or “patient” is meant any subject, preferably a mammalian subject, for whom diagnosis, prognosis, or therapy is desired. Mammalian subjects include humans, domestic animals, farm animals, sports animals, and zoo animals including, e.g., humans, non-human primates, dogs, cats, guinea pigs, rabbits, rats, mice, horses, cattle, and so on.

Terms such as “treat” or “treating” or “treatment” or “suppress” or “suppressing” or “alleviate” or “alleviating” refer to therapeutic measures that cure, slow down, lessen symptoms of, and/or halt progression of a diagnosed pathologic condition or disorder. Thus, those in need of treatment include those already with the disorder. In certain embodiments, a subject is successfully “treated” for a disease or disorder according to the methods provided herein if the patient shows, e.g., total, partial, or transient alleviation or elimination of symptoms associated with the disease or disorder.

“Prevent” or “prevention” refers to prophylactic or preventative measures that prevent and/or slow and/or reduce the incidence of the development of a targeted pathologic condition or disorder. Thus, those in need of prevention include those at risk of or susceptible to developing the disorder. Subjects that are at risk of or susceptible to developing a food allergy include, but are not limited to, subjects having a familial history of or a genetic marker for food allergy, subjects having a vitamin D deficiency, and obese subjects. In addition, subjects having one food allergy can be at risk for developing allergies to additional foods. In certain embodiments, a disease or disorder is successfully prevented according to the methods provided herein if the patient develops, transiently or permanently, e.g., fewer or less severe symptoms associated with the disease or disorder, or a later onset of symptoms associated with the disease or disorder, than a patient who has not been subject to the methods of the invention. In a prophylactic context, the UPR inhibitor can be administered at any time before or after an event that places a subject at risk of or susceptible to developing a food allergy, for example, exposure to a potentially allergenic food. In some aspects, the UPR inhibitor is administered prophylactically before the event. In some instances, the UPR inhibitor is administered prophylactically on the same day as the event. In either a treatment or prophylactic context, the methods can comprise administering antigen immunotherapy in addition to the UPR inhibitor. For example, the antigen can be derived from eggs, milk, peanuts, tree nuts, and/or fish. As used herein, “fish” refers to fin fish, and does not include shellfish. In embodiments in which antigen immunotherapy is administered, the antigen immunotherapy and the UPR inhibitor can be administered together at the same time, or separately at different times.

A “food allergy” or an “allergic reaction” is an immune-mediated response to an allergen, usually a protein, in food. Symptoms of an allergic reaction to food may include hives, eczema, nausea, vomiting, diarrhea, chest or stomach pain, nasal congestion, sneezing, coughing, tingling, itching, and/or swelling of the lips, tongue, and/or throat, difficulty swallowing, shortness of breath, wheezing, dizziness or fainting, rapid or thready pulse, drop in body temperature, loss of consciousness. Eosinophilic esophagitis, atopic dermatitis, and anaphylaxis are examples of conditions that may be caused by food allergy.

The term “pharmaceutical composition” refers to a preparation that is in such form as to permit the biological activity of the active ingredient to be effective, and which contains no additional components that are unacceptably toxic to a subject to which the composition would be administered. Pharmaceutical compositions can be in numerous dosage forms. Pharmaceutical compositions may comprise a pharmaceutically acceptable carrier, and can comprise one or more of a buffer (e.g. acetate, phosphate or citrate buffer), a surfactant (e.g. polysorbate), a stabilizing agent (e.g. human albumin), a preservative (e.g. benzyl alcohol), a penetration enhancer, an absorption promoter to enhance bioavailability and/or other conventional solubilizing or dispersing agents.

“Systemic administration” means that a pharmaceutical composition is administered such that the active agent enters the circulatory system, for example, via enteral, parenteral, inhalational, or transdermal routes. Enteral routes of administration involve the gastrointestinal tract and include, without limitation, oral, sublingual, buccal, and rectal delivery. Parenteral routes of administration involve routes other than the gastrointestinal tract and include, without limitation, intravenous, intramuscular, intraperitoneal, intrathecal, and subcutaneous.

An “effective amount” of a composition as disclosed herein is an amount sufficient to carry out a specifically stated purpose. An “effective amount” can be determined empirically, in relation to the stated purpose, route of administration, and dosage form.

Applicant has discovered that a UPR inhibitor may be used as a novel, safe, and effective treatment for food allergy. Disclosed herein are compositions and methods for preventing, suppressing, treating, or reducing the incidence of an allergic reaction.

A relatively small percentage of protein antigens are allergens. Compared to other antigens, allergens have a strong capacity to induce a type 2 cytokine response (IL-4, IL-5, IL-9, and IL-13), with these cytokines playing pathogenic roles in mouse models (Sicherer et al. 2010c; Morafo et al. 2003; Birmingham et al. 2007; Osterfeld et al. 2010; Berin et al. 2009). These cytokines induce food allergy by promoting IgE production, mastocytosis, eosinophilia, increased smooth muscle contractility, intestinal mastocytosis, and intestinal epithelial permeability (Finkelman et al. 1988; Sanderson 1988; Madden et al. 1991; Chen et al. 2015; Zhao et al. 2003; Madden et al. 2002). Recently, three cytokines, thymic stromal lymphopoietin (TSLP), IL-25, and IL-33, which are produced by epithelial cells located at interfaces between a vertebrate and its environment (Saenz et al. 2008; Paul et al. 2010), have been shown to act through multiple mechanisms on multiple cell types to promote a type 2 cytokine response (Paul et al. 2010). Accordingly, these are referred to collectively as “pro-Th2 cytokines.” Although allergenicity has been associated with some functional characteristics of antigens, such as protease activity, little is understood about common pathways that might connect these functional characteristics to induction of type 2 cytokine production.

Cellular stress can lead to the accumulation of unfolded or misfolded proteins in the endoplasmic reticulum. This activates an unfolded protein response (UPR) in affected cells, in which protein translation is halted, misfolded or unfolded proteins are degraded, and chaperone protein production is increased. Apoptosis results in when the cell's normal function cannot be restored by the UPR.

The instant disclosure provides a novel, first-in-class therapy for the treatment of food allergy. Applicant has discovered a connection between the UPR and the pro-Th2 cytokine response in food allergenicity. In particular, Applicant has shown that UPR plays a role in food allergy by inducing pro-Th2 cytokine (IL-25, IL-33, and TSLP) and UPR-related (PERK, BiP, CHOP, ATF4, ATF6, ERN1, XBP1, and XBP1s) gene expression. These results led to the surprising discovery that inhibition of the UPR is a highly differentiated therapy, which is efficacious across multiple food allergens, meeting the needs of a broad food allergy population.

Applicant has shown that 1) treatment with a blocking monoclonal antibody (mAb) to any of the pro-Th2 cytokines inhibits food allergy development; 2) treatment with a combination of all three pro-Th2 cytokine blocking mAbs during oral exposure of immunologically naïve mice to medium chain triglycerides plus egg white (MCT/EW) leads to egg white tolerance, instead of food allergy; 3) treatment with UPR inhibitors, such as metformin, 4-PBA, TUDCA, or blocking mAbs agains pro-Th2 cytokines suppresses established food allergy; 4) induction of food allergy in Applicant's system is accompanied by increases in lamina propria Th2 cells, mast cells, eosinophils, and dendritic cells, but not ILC2s; and 5) the increases in Th2 cell, mast cell, and eosinophil number are suppressed by anti-pro-Th2 cytokine mAb treatment.

Applicant's data suggest a novel mechanism that contributes to allergenicity: components of several allergenic foods stress epithelial cells, which respond by developing the stress-relieving UPR. One or more UPR signaling pathways and transcription factors then stimulate expression of the three pro-Th2 cytokines, which both induce and maintain food allergy. Remarkably, components of members of five of the nine most important classes of food allergens (lipid fractions of egg and milk, and aqueous extracts of peanuts, a tree nut (walnut), and a fish (codfish) induce UPR-associated and pro-Th2 cytokine gene expression in CACO-2 intestinal epithelial cells. Further, metformin, which suppresses the UPR by inducing AMP kinase, also suppresses pro-Th2 cytokine gene induction in every case. TUDCA and 4-PBA, which suppress the UPR differently from metformin by acting as chemical chaperonins for unfolded/misfolded proteins in the endoplasmic reticulum, suppress pro-Th2 cytokine gene induction by EYP and heavy cream (the only allergens tested for these inhibitors). UPR-associated and pro-Th2 cytokine genes were also induced in CACO-2 cells by purified saturated fatty acids. The sodium salt of palmitic acid, the saturated fatty acid that was studied most thoroughly, induced UPR-associated and pro-Th2 genes in human intestinal organoids as well as in CACO-2 cells, with suppression of this gene induction by metformin Similar results were observed in intestinal organoids when palmitate was replaced by EYP.

Pro-Th2 cytokine induction by EYP and by a purified fatty acid (palmitate) occurs at the protein as well as at the RNA level in cultured CACO-2 cells. EYP induces both UPR-associated and pro-Th2 cytokine gene expression in three epithelial organs at the interface between the host and its environment (skin, airways, and gut), and EYP induction of these genes is suppressible by metformin. The demonstration that metformin treatment suppresses development of food allergy, and that both metformin and TUDCA ameliorate established disease supports a correlation between development of UPR and induction and maintenance of at least some types of food allergy.

In one aspect, a method of suppressing an allergic reaction to food is disclosed. The method may comprise the step of administering to a subject with a food allergy a pharmaceutical composition comprising an inhibitor of UPR. In another aspect, the invention provides a method of preventing a food allergy, the method comprising administering to a subject susceptible to developing a food allergy a pharmaceutical composition comprising an inhibitor of UPR. In a further aspect, the invention provides a pharmaceutical composition for use in treating or preventing a food allergy, the pharmaceutical composition comprising an inhibitor of UPR. In an additional aspect, the invention provides the use of an inhibitor of UPR in the manufacture of a medicament for the treatment or prevention of a food allergy.

The food can be selected from the group consisting of eggs, milk, peanuts, tree nuts, and fish. In one embodiment, the allergic reaction is anaphylaxis.

In one embodiment, the inhibitor of UPR is metformin. In another embodiment, the inhibitor of UPR may be 4-phenylbutyrate (4-PBA). In an additional embodiment, the inhibitor of UPR may be a bile acid, for example, ursodeoxycholic acid (UDCA) or tauroursodeoxycholic acid (TUDCA), or a pharmaceutically acceptable salt thereof. In certain embodiments, the methods and uses of the invention involve pharmaceutical compositions comprising metformin and UDCA, or pharmaceutical compositions comprising metformin and TUDCA.

Embodiments of the present disclosure can be further defined by reference to the following non-limiting examples. It will be apparent to those skilled in the art that many modifications, both to materials and methods, can be practiced without departing from the scope of the present disclosure.

### EXAMPLES

### Example 1. Allergen-Induced Pro-Th2 Cytokine and UPR-Related Gene Expression is Suppressed by UPR Inhibitors in Cultured Human Intestinal Epithelial Cells

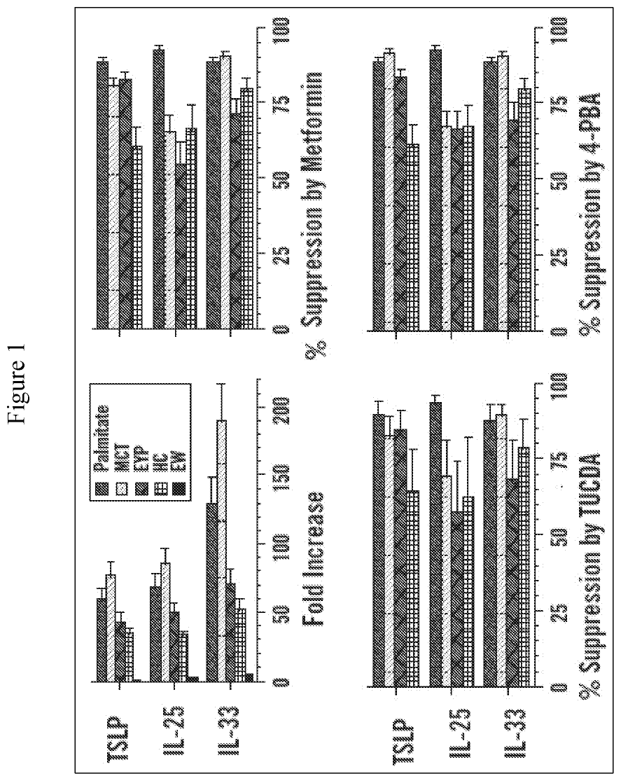

To evaluate whether the UPR induces pro-Th2 gene expression in cultured human intestinal epithelial cells, we evaluated the abilities of three UPR inhibitors to inhibit the ability of egg yolk plasma (EYP), heavy cream (HC), medium chain triglycerides (MCT), or sodium palmitate to induce pro-Th2 cytokine expression by CACO-2 cells. CACO-2 cells were cultured for 24 hours with medium alone, EW, negative control, EYP, HC, MCT, or sodium palmitate (positive control for UPR induction), with or without the UPR inhibitors metformin, TUDCA, or 4-PBA. mRNA was prepared, reverse-transcribed, and quantitated by real time PCR. Results show that EYP and HC, along with MCT and palmitate, induced increased expression of the pro-Th2 cytokines, with strong suppression by the UPR inhibitors (FIG. 1).

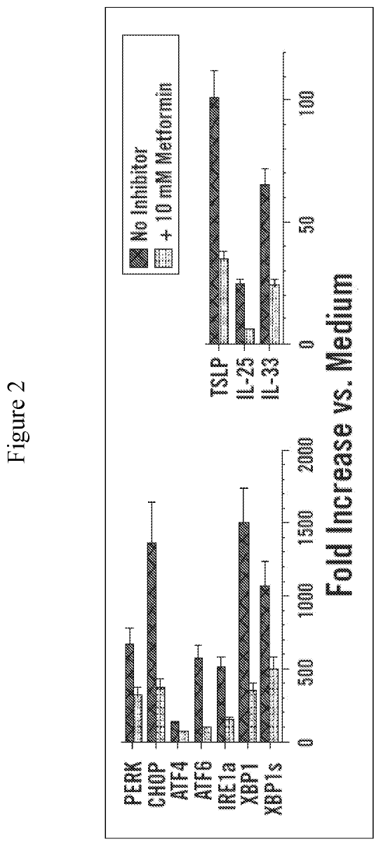

Because the UPR can be induced by multiple mechanisms that are associated with cell stress, including mechanisms that are not associated with lipids, Applicant investigated the ability of aqueous extracts of allergenic foods other than eggs and milk to induce UPR-associated and pro-Th2 cytokine gene expression, and the ability of metformin to suppress these responses. CACO-2 cells were cultured for 24 hours with medium alone or with a lipid-free peanut extract±metformin. mRNA was reverse-transcribed and gene expression was determined by real time PCR. Data show that peanut extract increased the expression of UPR-related genes (FIG. 2, A) and pro-Th2 cytokine genes (FIG. 2, B) by CACO-2 cells, and that pro-Th2 cytokine gene expression is UPR-dependent.

### Example 2. Multiple Mechanisms Lead to Allergen-Induced Expression of Pro-Th2 Cytokine and UPR-Related Genes

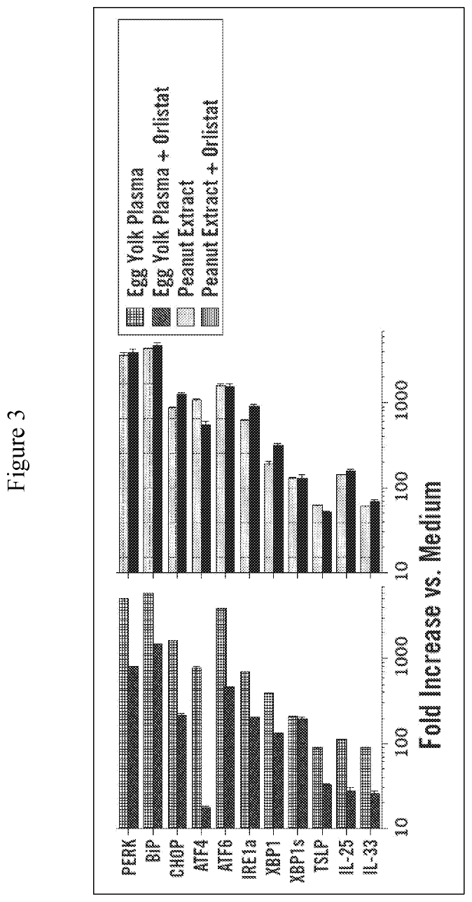

CACO-2 cells were cultured for 24 hours with medium alone, egg yolk plasma, or peanut extract, with or without the lipase inhibitor Orlistat. mRNA was extracted and reverse transcribed, after which UPR-associated gene expression and pro-Th2 cytokine gene expression were determined by real time PCR. Data show that the lipase inhibitor suppressed the egg yolk plasma-induced gene expression (FIG. 3). This is because triglycerides must be hydrolyzed into glycerol and free fatty acids to allow fatty acid absorption. In contrast, the lipase inhibitor had no effect on UPR-related or pro-Th2 gene expression by peanut extract. These results show that peanut-extract induction of UPR-related and pro-Th2 cytokine gene expression is not due to triglyceride contamination, and that peanut extract must induce the UPR through a mechanism different from that used by egg yolk plasma.

### Example 3. Increased Pro-Th2 Cytokine Gene Expression Results in Increased Pro-Th2 Cytokine Production In Vitro

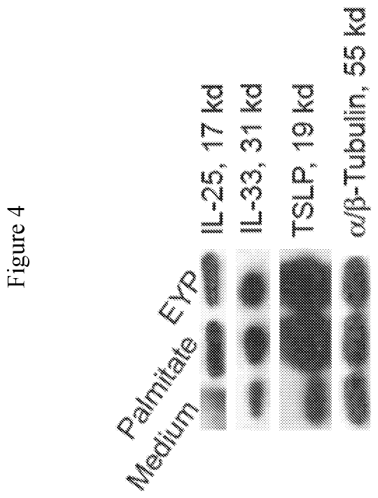

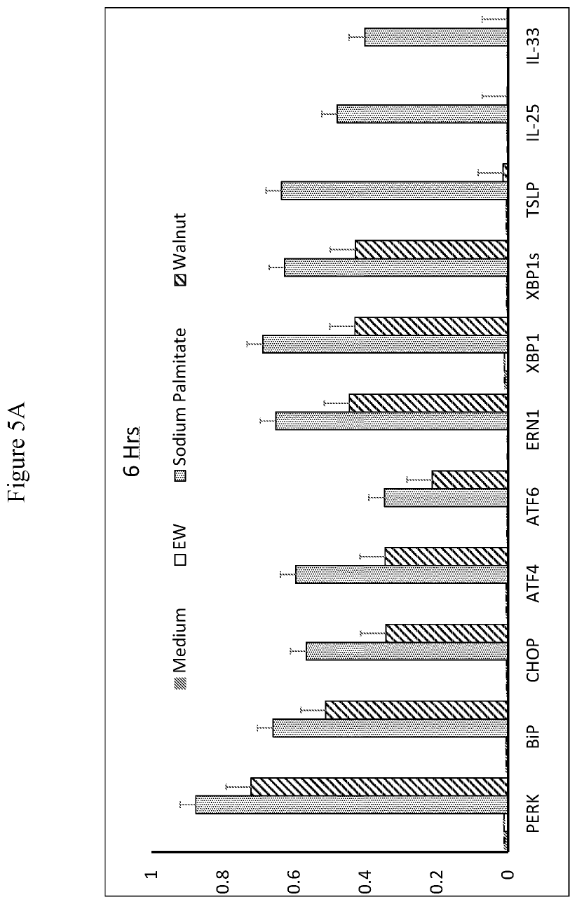

CACO-2 cells were cultured for 24 hr±palmitate or EYP. Cell lysates were prepared and normalized by protein concentration and direct α/β-tubulin Western blot. Lysates were serially incubated with anti-IL-25+protein G beads, anti-IL-33+protein G beads, and anti-TSLP+protein G beads. Laemmli buffer eluates from beads were analyzed by electrophoresis on a 4-20% polyacrylamide gel, blotted onto a PVDF membrane, and visualized by incubation with biotinylated anti-IL-25, anti-IL-33, or anti-TSLP mAb, followed by streptavidin-peroxidase and ECL WB substrate. Results are shown in FIG. 4. These data show that egg yolk plasma increased pro-Th2 cytokine protein expression, not just gene expression.

### Example 4. Induction of Pro-Th2 Cytokine and UPR-Related Gene Expression by Additional Allergens

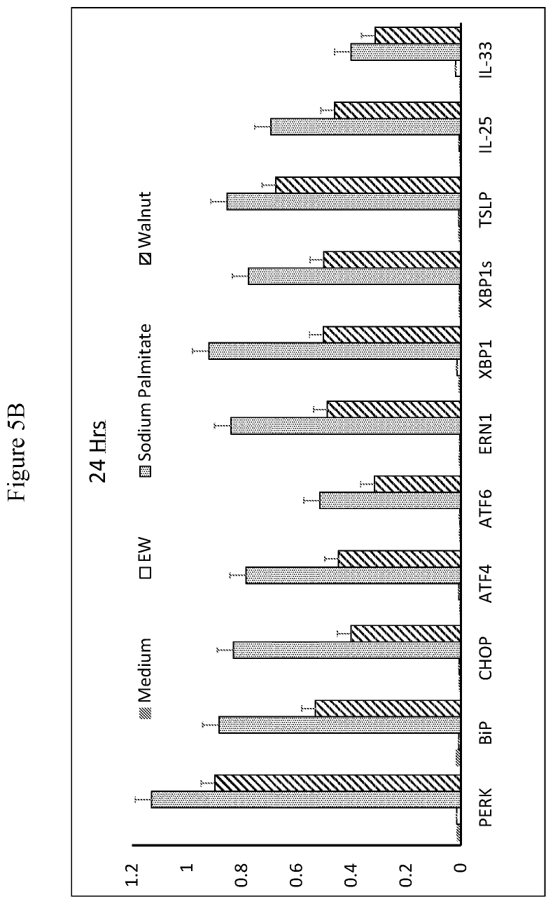

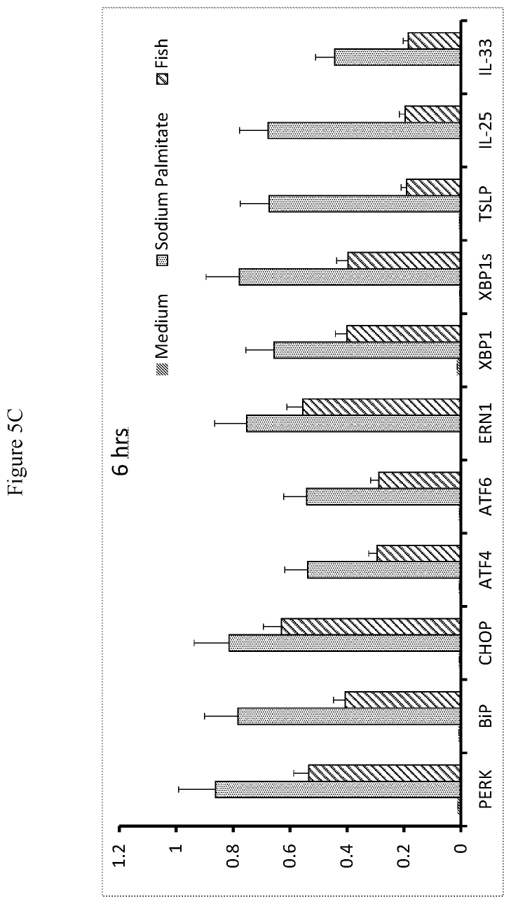

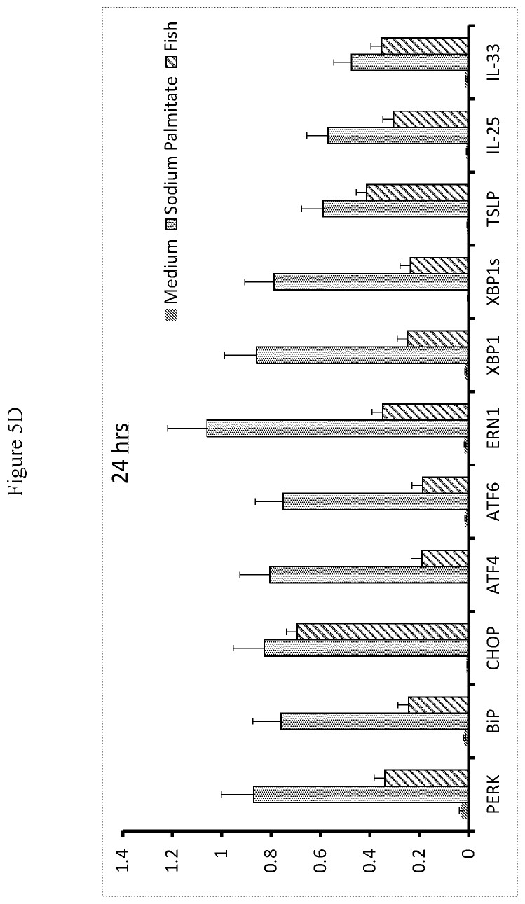

CACO-2 cells were cultured with medium alone, egg white (EW, a negative control), sodium palmitate (a positive control) or aqueous extracts of walnuts or codfish. Six and 24 hours later, cells were harvested, their RNA was extracted and reverse transcribed, and UPR-associated gene and pro-Th2 cytokine gene expression were determined by quantitative (real time) PCR. Results are shown in FIG. 5A-5D. These data indicate that walnuts resemble egg yolk plasma, milk fat, and peanut extract in inducing both the UPR and the pro-Th2 cytokine response, with the former preceding the latter.

### Example 5. UPR Inhibitors Suppress Pro-Th2 Cytokine and UPR-Related Gene Expression in Human Intestinal Organoids

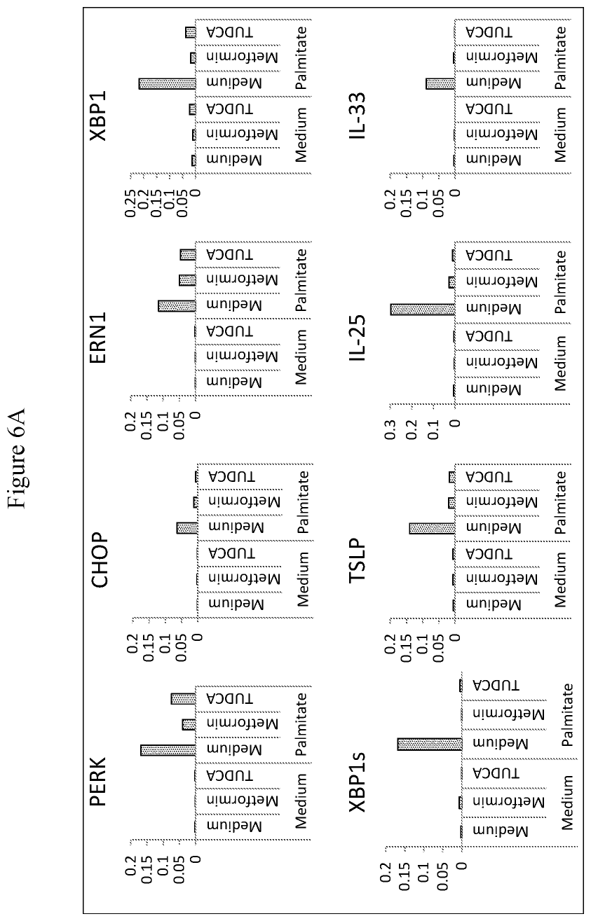

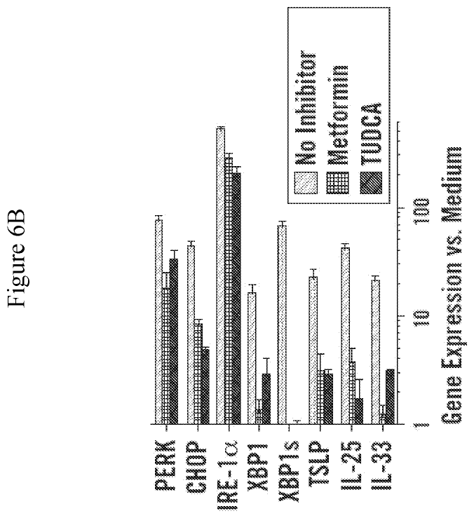

To evaluate the ability of UPR inhibitors to suppress pro-Th2 cytokine gene expression in non-transformed human cells that more closely resemble intestine, Applicant generated hollow human intestinal organoids (HIO), which have an epithelial cell lining and a mesenchymal cell exterior. HIO were produced from human peripheral blood pluripotent stem cells having a single UPR-associated gene deleted by CRISPR/Cas9. Applicant injected the lumens of HIO with culture medium or palmitate to induce increased UPR-associated and pro-Th2 cytokine gene expression, and further injected the HIO lumens with metformin or TUDCA. mRNA levels for the UPR-associated and pro-Th2 cytokine genes were determined by real-time PCR. Data show that the UPR inducer sodium palmitate stimulated UPR-dependent induction of pro-Th2 cytokine genes by the non-transformed human organoids, and that UPR inhibitors suppressed palmitate-induced gene expression (FIG. 6A-6B).

### Example 6. IRE-1α is Important for UPR Induction of Pro-Th2 Cytokine Expression In Vitro

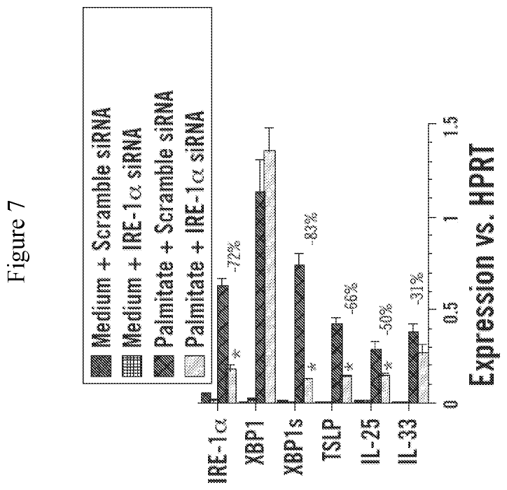

Applicant evaluated the ability of small inhibitory (si) RNA that specifically inhibits the UPR signaling molecule, IRE-1α, to suppress pro-Th2 cytokine expression by palmitate-stimulated CACO-2 cells. IRE-1α both induces the NF-ϰB and JAK signaling pathways and catalyzes the conversion of XBP-1 to the active transcription factor, XBP-1s. CACO-2 cells (6 wells/group) were cultured for 48 hours with 25 pM of GAPDH siRNA, scrambled siRNA, or IRE-1α siRNA. Sodium palmitate or medium was added to wells after 24 hours. RNA was extracted from harvested cells after 48 hours. Gene expression was quantitated by real-time PCR. IRE-1α siRNA significantly suppressed the palmitate-induced CACO-2 cell IRE-1α, XBP-1s, TSLP, and IL-25 responses and demonstrated a trend towards suppression of the IL-33 response (FIG. 7). In particular, GAPDH siRNA suppressed GAPDH expression by 83%, without significant effect on IRE-1α, XBP1, XBP1s, or pro-Th2 cytokine expression. IRE-1α siRNA suppressed IER-1α expression by 72% (p<0.05), XBP1s expression by 83% (p<0.05), TSLP by 66% (p<0.05), IL-25 by 50% (p<0.05), and IL-33 by 31% (NS).

### Example 7. Suppression of Pro-Th2 Cytokine Expression Prevents Development of Food Allergy

A previous study demonstrated that inoculation of mice with food (peanuts or ovalbumin) along with a common food constituent and additive, medium chain triglycerides (MCT), induces IgE-dependent peanut or ovalbumin food allergy, respectively, without requiring priming through a non-enteric route or the use of a conventional adjuvant (Li et al. 2013). Studies of the mechanisms involved in food allergy induction by this protocol demonstrated that MCT ingestion increases intestinal epithelial permeability as well as intestinal epithelial expression of each of the pro-Th2 cytokine genes (Li et al. 2013). This study did not, however, test whether any or all of the pro-Th2 cytokines were required for disease induction or maintenance in this system. Applicant have now used the food allergy model to test the roles of each pro-Th2 cytokine in disease pathogenesis. Applicant's results indicate that disease induction in this model can be blocked by inhibiting any of the pro-Th2 cytokines.

Pro-Th2 cytokine antagonists have a lasting effect on development of food allergy.

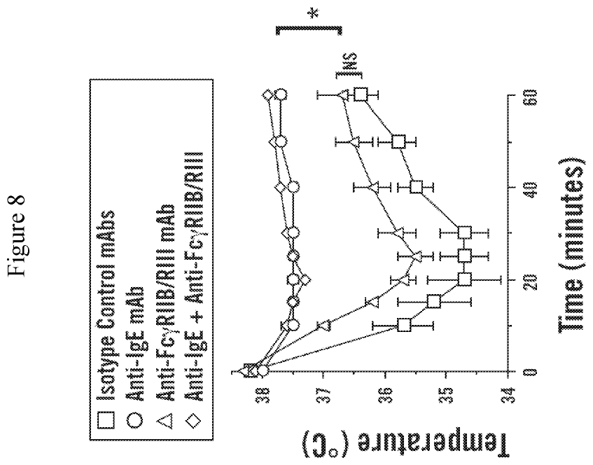

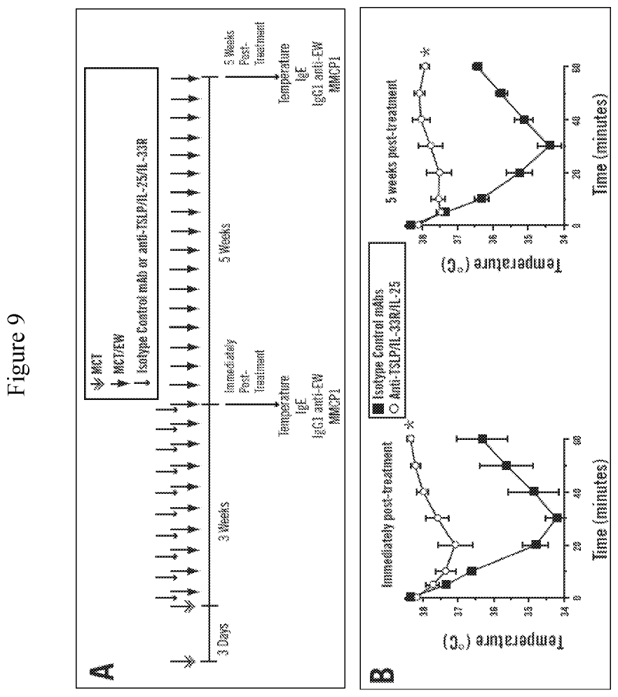

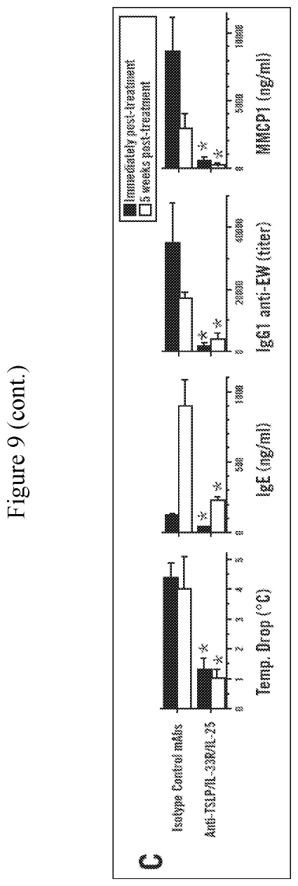

To determine whether Applicant's MCT/ovalbumin model of food allergy could be inhibited by systemic treatment with a combination of neutralizing monoclonal antibodies (mAbs) to all of the pro-Th2 cytokines, Applicant inoculated BALB/c female mice by o.g. with MCT on days 0 and 3, then o.g. every other day with an MCT/EW emulsion. Mice in one group also received i.p. injections of a combination of anti-IL-25, anti-IL-33R, and anti-TSLP mAbs 12 hours before each o.g. inoculation with MCT or MCT/EW, while mice in the other group were injected i.p. with isotype-matched control mAbs (FIG. 9A). After 3 weeks, mice that had received isotype control mAbs experienced an ˜4° C. drop in rectal temperature by 30 min after oral gavage with MCT/EW, which was shown in a separate experiment to be IgE-dependent (FIG. 8), while the temperature drop following oral challenge was ˜1.2° C. in mice that had been treated with the anti-pro-Th2 mAb cocktail (FIG. 9B). This suppressive effect reflected a >10-fold decrease in serum levels of MMCP1,which reflects mucosal mast cell degranulation (Strait et al. 2002) and IgG1 anti-EW Ab, as well as an ˜3-fold decrease in total serum IgE levels. This suppressive effect was persistent: when these mice were inoculated o.g. with EW/MCT for an additional 5 weeks in the absence of mAb injections, the mice that had initially been treated with anti-pro-Th2 mAbs continued to show considerable suppression of development of shock and IgG1, IgE, and MMCP1 responses (FIG. 9C).

IL-25, IL-33 and TSLP are all required for development of food allergy in EW+MCT-inoculated mice.

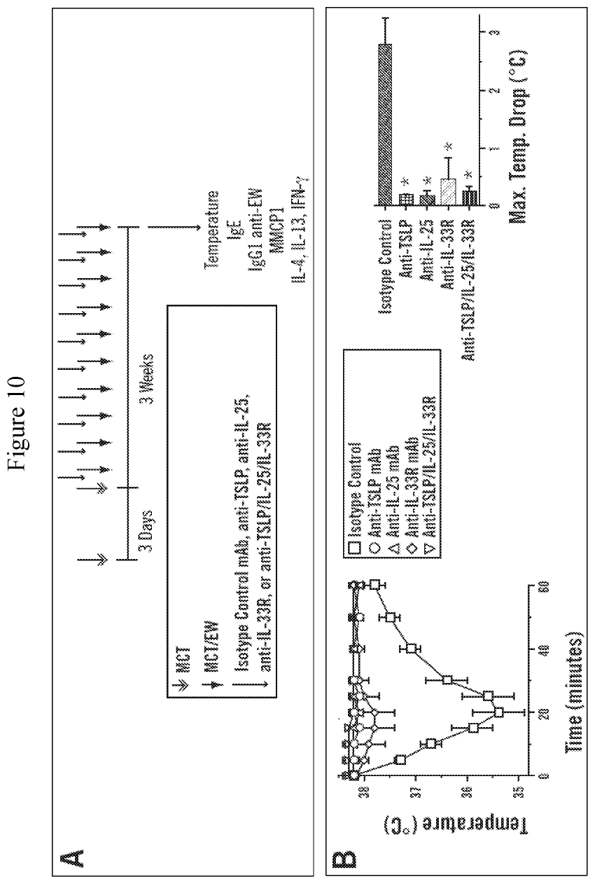

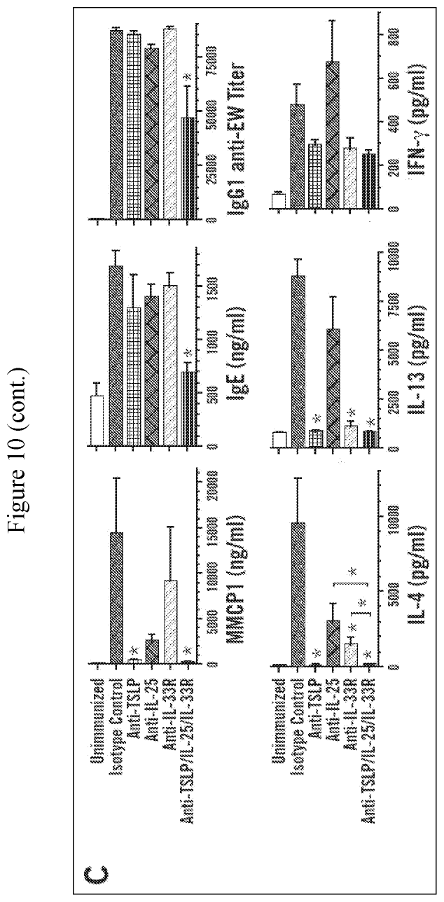

To determine which of the pro-Th2 cytokines are required for development of food allergy in Applicant's model, mice were not immunized or were inoculated o.g. with MCT, then EW/MCT, as in Applicant's initial experiment and were treated i.p. with isotype control mAbs, anti-TSLP, anti-IL-25, or anti-IL-33R mAb, or a combination of all 3 of these mAbs (FIG. 10A). After 3 weeks of this treatment, shock (>1° C. of hypothermia) in response to EW/MCT challenge developed in mice treated with the control mAbs, but not in mice treated with any of the anti-pro-Th2 cytokine mAbs (FIG. 10B). Suppression of development of shock (hypothermia) was complete in mice treated with anti-TSLP mAb, anti-IL-25 mAb, or with the mAb cocktail, while a small temperature drop was seen in anti-IL-33R mAb-treated mice. Anti-TSLP mAb suppressed IL-4 and IL-13 responses to basal levels and was more effective than either anti-IL-25 or anti-IL-33R mAb at suppressing the IL-4 and MMCP1 responses (FIG. 10C). Anti-TSLP and anti-IL-33R mAbs were more effective than anti-IL-25 mAb at suppressing IL-13 production. The mAb cocktail was slightly more effective than any of the single mAbs at suppressing the MMCP1 response, but otherwise resembled anti-TSLP mAb in its effects; there was a non-significant trend towards decreased MMCP1 in anti-IL-25 and anti-IL-33 mAb-treated mice. Importantly, the effects of the anti-pro-Th2 cytokines resulted from suppression of the Th2 response without a corresponding shift to a Th1 response, as judged from the lack of a significant increase in IFN-γ secretion in anti-pro-Th2 cytokine mAb-treated mice (FIG. 10C). Serum IgG1 anti-EW and IgE levels were only decreased significantly in mice that had received all 3 anti-pro-Th2 cytokine mAbs; the decreased IgE levels were similar to those in unimmunized mice, but IgG1 anti-EW Ab levels were still increased ˜5,000-fold above those in unimmunized mice (FIG. 10C).

### Example 8. Suppression of Pro-Th2 Cytokine Expression Suppresses Established Food Allergy

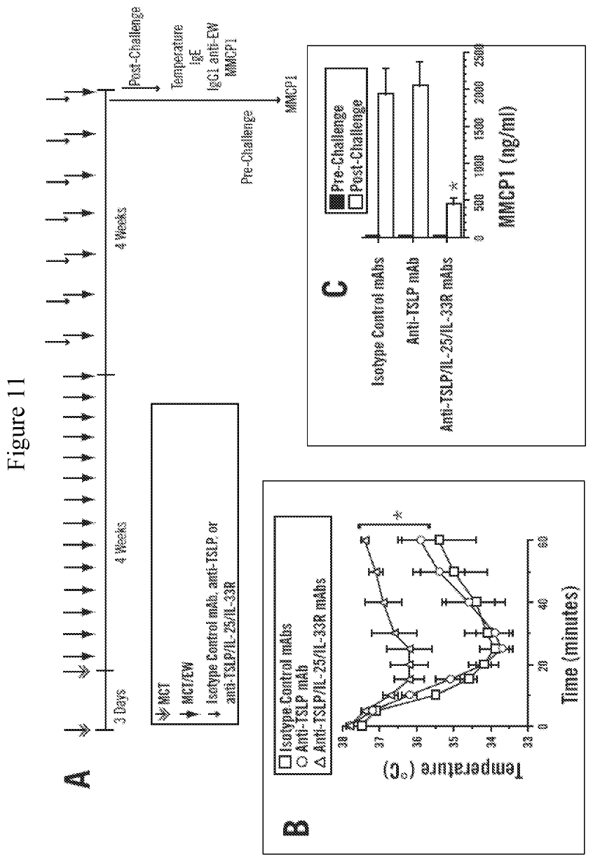

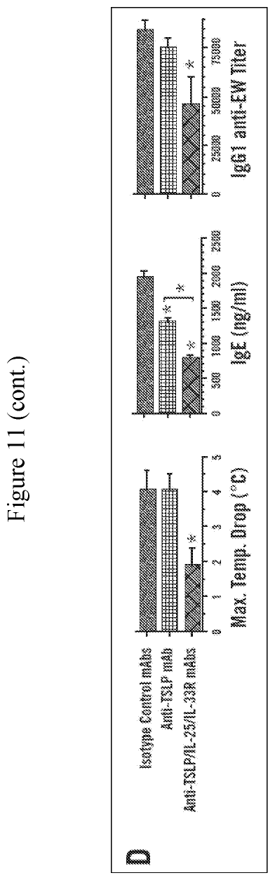

Because induction of Applicant's model of food allergy was most effectively suppressed by either anti-TSLP mAb or by a cocktail of all 3 anti-pro-Th2 cytokine mAbs, Applicant evaluated the ability of each of these mAb treatments to suppress food allergy that had been established by o.g. inoculation of mice with MCT, then EW/MCT for a total of 4 weeks prior to the initiation of mAb treatment (FIG. 11A). Mice were then inoculated o.g. with MCT/EW for an additional 4 weeks, but also received one of the i.p. mAb treatments. At the end of this 4-week treatment period, the hypothermia response to EW/MCT oral challenge was not affected by anti-TSLP mAb by itself, but was considerably suppressed by the mAb cocktail (FIG. 11B). In the same experiment, the MMCP1 response to MCT/EW challenge was not affected by anti-TSLP mAb alone, but was suppressed by ˜80% by the mAb cocktail (FIG. 11C); the cocktail was also more effective than anti-TSLP mAb alone at suppressing serum IgE and IgG1 anti-EW Ab levels (FIG. 11D).

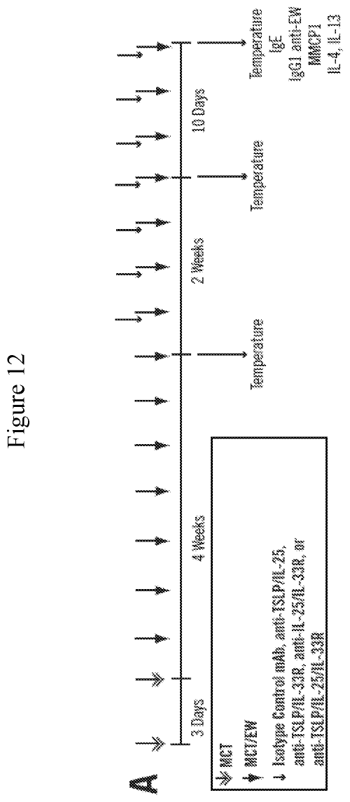

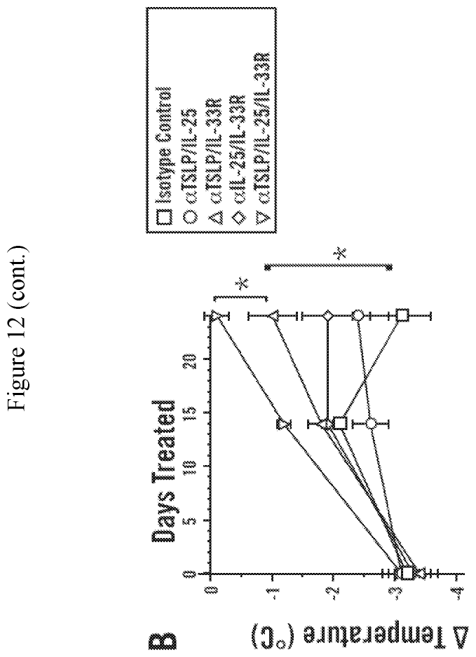

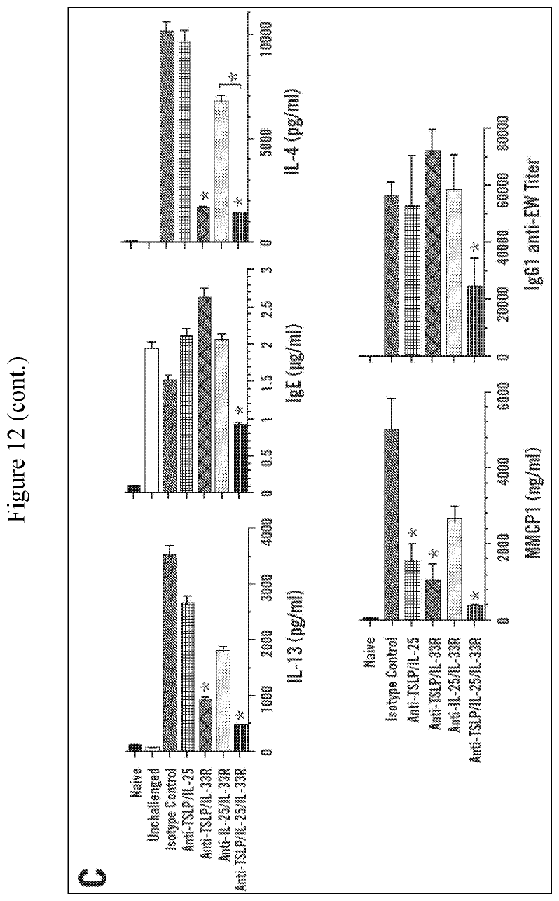

In an additional experiment with mice that were induced to develop food allergy prior to the initiation of mAb treatment (FIG. 12A), 24 days of treatment with the mAb cocktail totally suppressed the development of shock (FIG. 12B) and decreased the MMCP1 response to oral challenge by >90%. The same treatment decreased IL-4 and IL-13 responses to oral challenge by 80-90% and total serum IgE and IgG1 anti-EW Ab levels by ˜50% (FIG. 12C). A combination of anti-TSLP and anti-IL-33R mAbs showed less complete ability to suppress food allergy in this time frame, while combinations of anti-TSLP and anti-IL-25, or anti-IL-25 and anti-IL-33R mAbs were even less effective (FIG. 12C).

### Example 9. Maintenance of Cellular Changes in Food Allergy Is Pro-Th2 Cytokine-Dependent

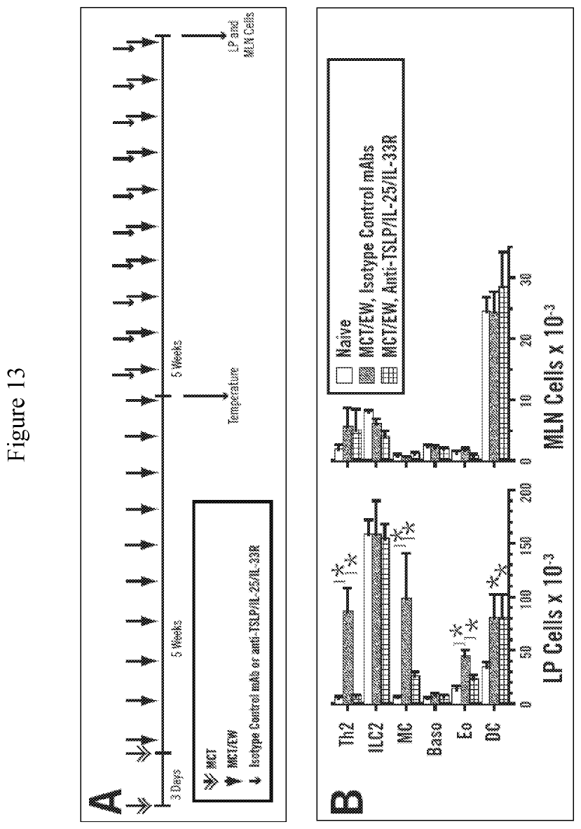

To evaluate the cellular changes that accompany the development of food allergy in Applicant's model, Applicant inoculated mice twice a week o.g. for 5 weeks to induce food allergy (defined as a temperature drop>2° C. in response to o.g. challenge), then continued these o.g. inoculations for an additional 5 weeks, but injected mice i.p. with all 3 anti-pro-Th2 cytokine mAbs or isotype control mAbs 4 hours before each o.g. inoculation (FIG. 13A). At the end of this 10-week period, control mAb-treated mice, but not anti-pro-Th2 cytokine mAb-treated mice continued to develop hypothermia in response to o.g. MCT/EW. Studies of lamina propria and MLN cells obtained at this time showed large, significant increases in numbers of Th2 cells and mast cells, and smaller significant increases in numbers of eosinophils and dendritic cells in the isotype control mAb-treated mice (FIG. 13B). No increases in ILC2 were observed in treated mice, as compared to untreated mice. Treatment with the cocktail of anti-pro-Th2 cytokine mAbs suppressed the increases in lamina propria Th2 cell, mast cell, and eosinophil number, but not the increase in dendritic cell number. Induction of food allergy did not significantly increase any of these cell populations in MLN (FIG. 13B).

### Example 10. UPR Inhibitors Suppress Pro-Th2 Cytokine and UPR-Related Gene Expression In Vivo

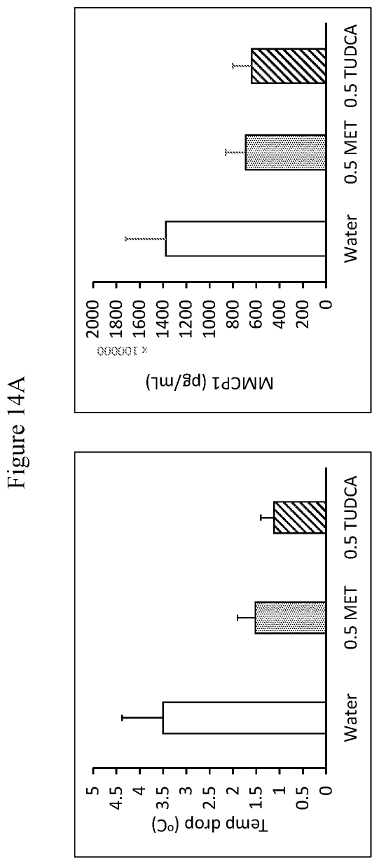

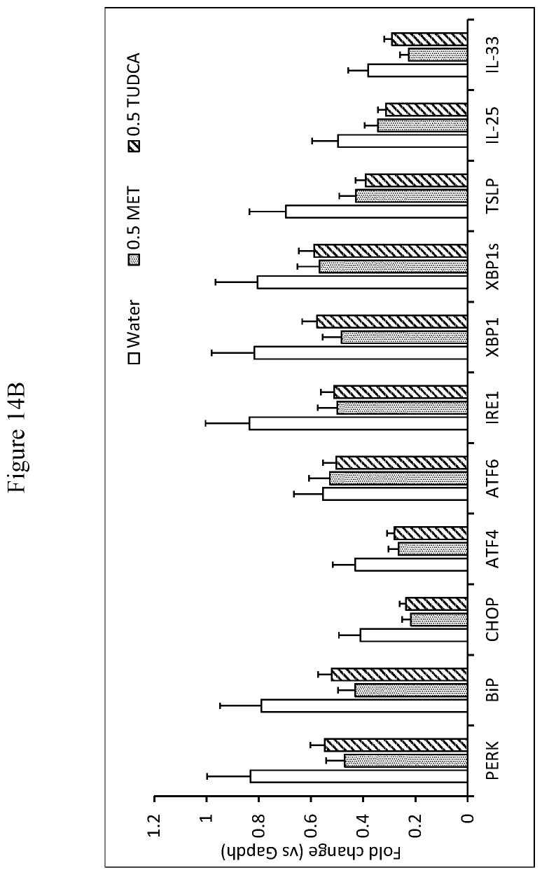

BALB/c mice were induced to develop food allergy and were then treated with 500 mg/kg doses of metformin or TUDCA in drinking water. Results show suppression by UPR inhibitors of hypothermia (FIG. 14A), mouse mast cell protease 1 (MMCP1) response (FIG. 14B), and small intestinal UPR-related gene expression (except ATF6) and pro-Th2 cytokine gene expression (FIG. 14C).

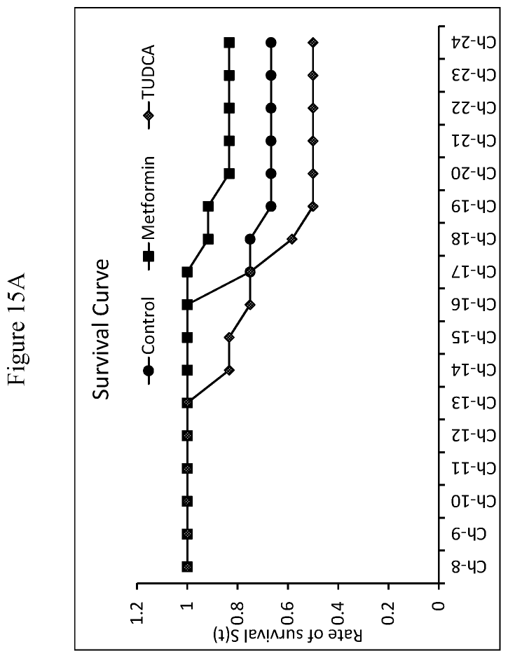

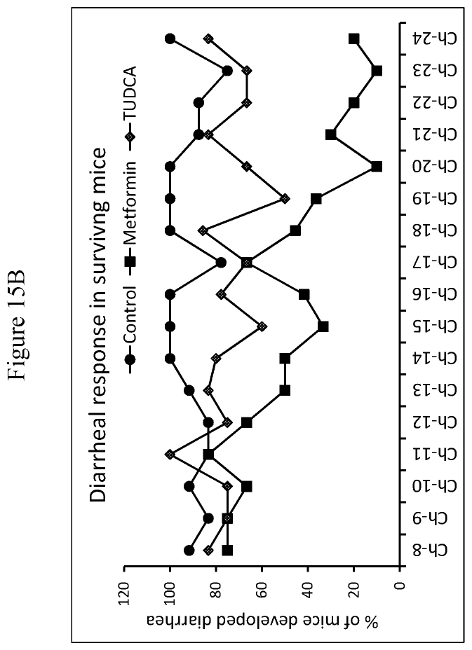

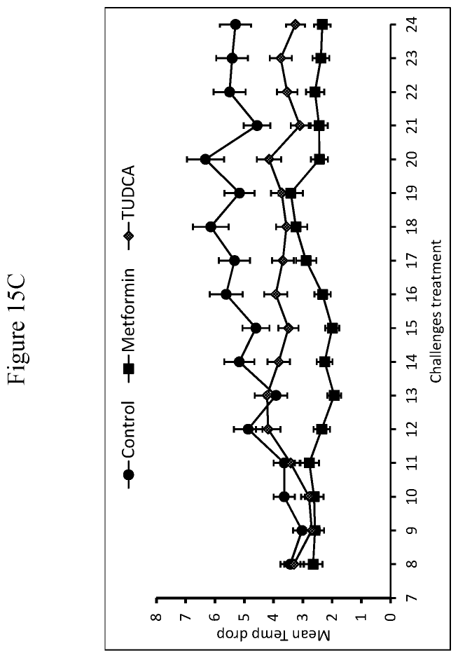

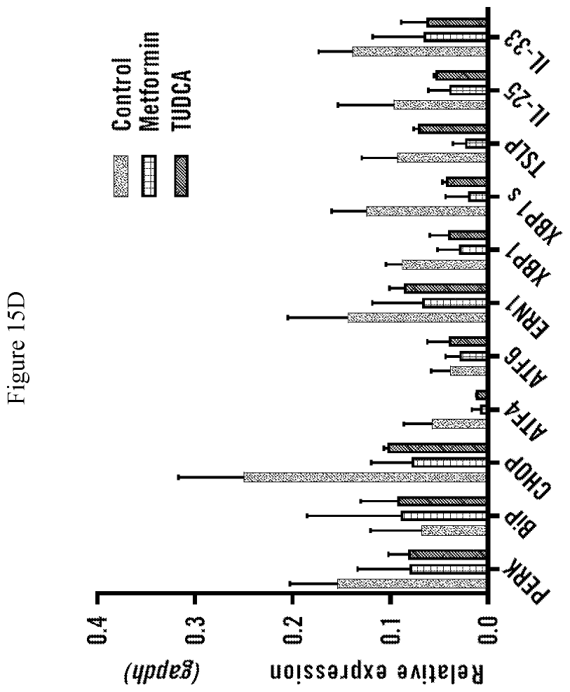

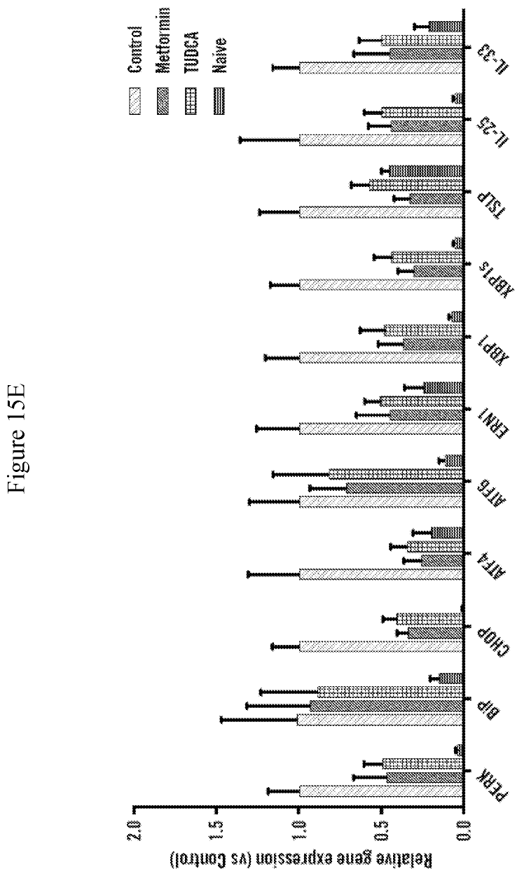

BALB/c mice with established egg allergy were provided with drinking water that contained 500 mg/kg/day of metformin or TUDCA, or with ordinary drinking water. Results are shown in FIG. 15A-15E. Both metformin and TUDCA suppressed intestinal UPR-related gene expression (except for BiP and ATF6) and pro-Th2 cytokine expression. Note the large increases in intestinal UPR-associated and pro-Th2 gene expression compared to naïve mice, and the partial suppression of UPR-associated and pro-Th2 genes by metformin and TUDCA (FIG. 15E).

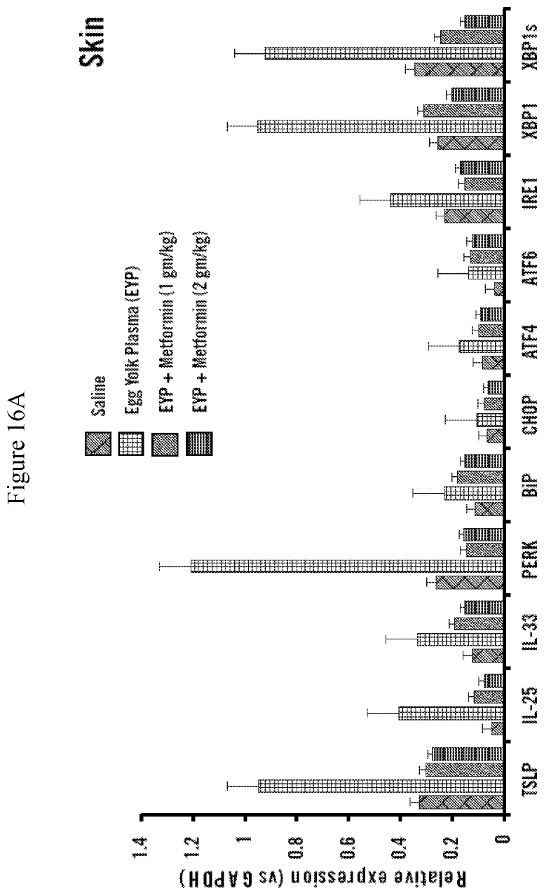

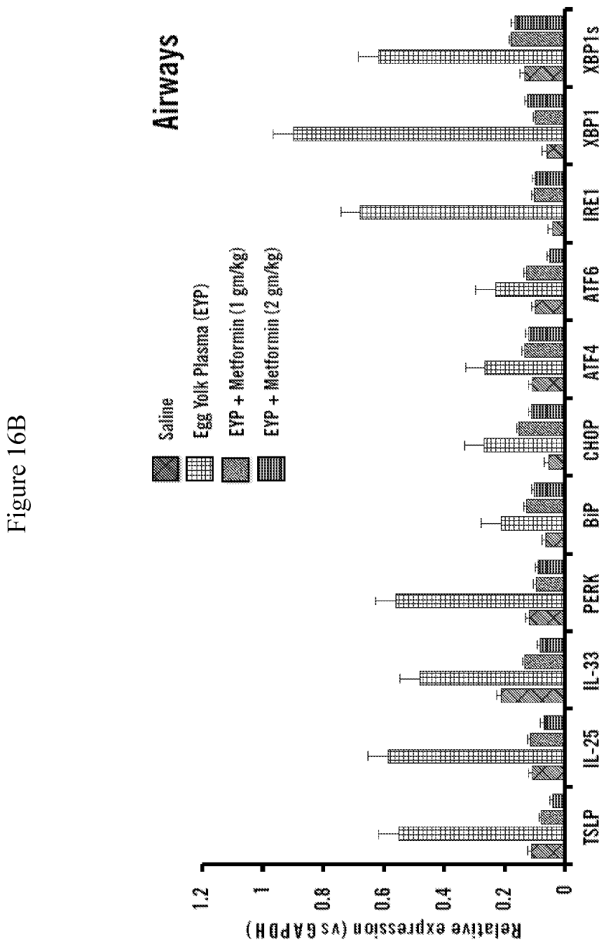

BALB/c mice with established egg allergy were administered egg yolk plasma with or without 1 g/kg/day or 2 g/kg/day of metformin. Pro-Th2 cytokine and UPR-related gene expression was inhibited in skin (FIG. 16A) and lung (FIG. 16B).

### Example 11. Representative Materials and Methods

In Vitro Studies

CACO-2 cells were obtained from the American Type Culture Collection (ATCC), catalog number HTB-37. These cells were grown in ATCC-formulated Eagle's Minimum Essential Medium (EMEM) (Cat. No. 30-2003), supplemented with fetal bovine serum (FBS) to a final concentration of 20% (ATCC® Cat. No. 30-2020) and Gibco antibiotic-antimycotic mixture. The cells were cultured in 6-well, 12-well, or 24-well culture plates to approximately 85%-90% confluency.

On the day of experiment, EMEM medium was prepared with the appropriate concentrations of stimulants and/or inhibitors and added to the cells. Following the period of culture (usually 6 or 24 hours), the cells were washed and lysed, and total RNA was purified using PureLink™ RNA Mini Kit (Thermo Fisher Cat. No. 12183018A). The purified total RNA was used to generate cDNA using a High-Capacity cDNA Reverse Transcription Kit (Applied Biosystems, Cat. No. 4368814). Gene expression was analyzed using the qPCR method with a BioRad My iQ™ Detection system, and compared to the expression of the GAPDH housekeeping gene.

Mice. Seven- to eight-week old BALB/c female mice were purchased from the NCI Animal work was approved by the Cincinnati Children's Hospital Research Foundation IACUC.

Reagents. Medium chain triglycerides (MCT) (Nestle Health Science, Switzerland) were purchased at a local pharmacy. Anti-IL-33R mAb (which binds to the long form of ST2, the receptor for IL-33) and anti-IL-25 mAb (clone 2C3, originally produced in the Andrew McKenzie laboratory, Cambridge, UK) were obtained from Janssen pharmaceuticals. 28F12, a hybridoma that produces anti-TSLP mAb was a gift of Dr. Andrew Farr, University of Washington. Egg white (EW) removed sterilely from organic hen's eggs was dialized against double distilled water and centrifuged for 20 min at 3,900 rcf. The supernatant was concentrated with a stirred ultrafitration cell unit (Millipore, USA) with a 10 kDa Diaflo membrane. Protein concentration was evaluated with a BCA protein assay kit (Pierce, USA) according to the manufacturer's protocol.

Immunofluorescence and Flow Cytometry. To identify cell types among lamina propria (LP) and mesenteric lymph node (MLN) cells, single cell suspensions prepared from these tissues were first stained with phycoerythrin (PE)-conjugated anti-c-Kit (Biolegend, clone 2B8), PE-Cy7-conjugated anti-FcεRIα (Biolegend, clone MAR-1), allophycocyanin (APC)-conjugated anti-IL17RB, fluorescein isothiocyanate (FITC)-conjugated anti-β7 Integrin (BD Biosciences, clone M293), V500-conjugated anti-CD4 (BD Biosciences, clone RM4-5) and APC-Cy7-conjugated anti-CD3 (Biolegend, clone 145-2C11). Subsequently, cells were counterstained with PerCP-Cy5.5-conjugated monoclonal antibodies against lineage (Lin) markers CD8α (Biolegend, clone 53-6.7), B220 (Biolegend, clone RA3-6B2), CD11c (BD Biosciences, clone HL3), and Gr-1 (BD Biosciences, clone RB6-8C5). For identifying dendritic cells, LP cells or MLN cells were first stained with PE-conjugated anti-MHC class II (ebioscience, clone NIMR-4), APC-Cy7-conjugated anti-CD11c (ebioscience, clone NIMR-4), FITC-conjugated anti-CD103 (BD Biosciences, clone M290), Pacific Blue-conjugated anti-CD11b (BD Biosciences, clone M1/70), V500-conjugated anti-Gr-1 (Biolegend, clone RB6-8C5), PE-Cy7 conjugated anti-CD3 (BD Biosciences, clone 145-2C11), APC-conjugated anti-CX3CR1 (R&D Systems), and biotinylated antibodies against lineage markers Ter119 and CD19 (BD Biosciences, clones TER-119 and 1D3 respectively). Subsequently, cells were counterstained with PE-Cy7-labeled streptavidin (BD Biosciences). After staining, the cells were analyzed with a FACS Canto II (BD Biosciences). The following cell types were identified by the following surface markers and light scatter characteristics: Th2 cells: c-kit, FcεR1α⁻, ST2⁻, CD19⁻, Ter110⁻, CD3⁺, CD4⁺, IL17RB⁺, lymphocyte gates for forward and side scatter; ILC2 cells: c-kit⁻, FcεR1α⁻, CD19⁻, Ter110⁻, CD3⁻, CD4⁻, IL17RB⁻, ST2+, lymphocyte gates for forward and side scatter; mast cells (MC): CD19⁻, Ter110⁻, CD3⁻, CD4⁻, IL17RB⁻, c-kit⁺, FcεR1α⁺, high side scatter; basophils: CD3⁻, B220⁻, Gr1⁻, CD11c⁻, IL-17RB⁻, c-kit⁻, FcεR1α⁺, intermediate forward and side scatter; eosinophils: CD3⁻, CD19⁻, Ter119⁻, Gr1^{intermediate}, CD11c⁻, MHCII⁻, CD11b⁺, high side scatter; and dendritic cells (DC): CD3⁻, CD19⁻, Ter119⁻, Gr1⁻, CD11c⁺, MHC Class II⁺.

Induction of Food Allergy. Mice were inoculated with 0.1 ml of MCT by oral gavage (o.g.) through an 18-gauge needle with a spherical tip on day 0 and day 3, then inoculated o.g. with an emulsion (produced by thorough mixing, followed by brief sonication) of 100 μl of MCT and 100 mg of EW (total volume, 400 μl), as specified in the protocols shown in the figures. Mice were fasted for 4 hours before each oral treatment.

Pro-TH2 Cytokine Blockade. IL-25, IL-33, and TSLP were blocked systemically by intraperitoneal (i.p.) injection of mice with the corresponding mAbs 4 or 12 hours before each MCT or MCT/EW treatment. The quantities of blocking mAbs/week/mouse were based on preliminary studies that identified the doses required to block in vivo function: anti-TSLP, 0.5 mg; anti-IL-33R, 0.1 mg; anti-IL-25, 0.5 mg.

Measurement of IL-4, IL-13, IFN-γ, antigen-specific IgG, IgE, and mouse mast cell protease 1. In vivo IL-4 and IFN-γ cytokine secretion were measured by in vivo cytokine capture assay (IVCCA) as previously described (Finkelman et al. 2003; Finkelman et al. 1999). In vivo secretion of IL-13 was measured by a similar procedure, except that mice were injected with 2 μg of biotin-labeled anti-IL-13 mAb (clone 54D1) and ELISA wells were coated with anti-IL-13 mAb 53F5 (both mAbs were obtained from AbbVie (North Chicago, Ill.)). EW-specific IgG1 was measured by an ELISA in which ELISA plates (Costar, USA) were coated with EW (10 μg/ml) overnight, then washed and loaded with serial dilutions of mouse sera. After washing, wells were sequentially loaded with 1 μg/ml of biotin-anti-mouse IgG1 (eBioscience, USA) followed by 100 ng/ml of HRP-streptavidin and SuperSignal ELISA substrate, Peroxide and Enhancer solution diluted 20-fold in 20 mM Tris-Saline pH 7.2 (Pierce Biotechnology). Serum levels of MMCP-1 and IgE were measured with the corresponding ELISA kits (eBioscience, USA) according to the manufacturer's protocols.

Analphylaxis. The severity of anaphylactic shock was assessed by change in rectal temperature measured by digital thermometry (Strait et al. 2002; Dombrowicz et al. 1997).

Statistics. Differences in temperature and concentrations of MMCP-1, IL-4, IL-13, IFN-γ, IgE, and IgG1 anti-EW Ab were compared using Student's t test (GraphPad Prism 4.0; GraphPad software). A one-tailed test was used to test hypotheses that MCT/EW immunization would increase the parameters studied, that an anti-pro-Th2 cytokine mAb or mAbs would decrease these parameters, and that increasing the number of anti-pro-Th2 cytokine mAbs used would further decrease these parameters. A 2-tailed t test was used to compare cell numbers (FIG. 13B). For all experiments, a p value<0.05 was considered significant.

### REFERENCES

Berin M C, Mayer L. Immunophysiology of experimental food allergy. Mucosal Immunol 2009; 2:24-32.

Birmingham N P, Parvataneni S, Hassan H M, Harkema J, Samineni S, Navuluri L, et al. An Adjuvant-Free Mouse Model of Tree Nut Allergy Using Hazelnut as a Model Tree Nut. Int Arch Allergy Immunol 2007; 144:203-10.

Branum A M, Lukacs S L. Food allergy among children in the United States. Pediatrics 2009; 124:1549-55.

Chen C-Y, Lee J-B, Liu B, Ohta S, Wang P-Y, Kartashov A, et al. Induction of interleukin-9-producing mucosal mast cells promotes susceptibility to IgE-mediated experimental food allergy Immunity 2015; 43:788-802.

Dombrowicz D, Flamand V, Miyajima I, Ravetch J V, Galli S J, Kinet J P. Absence of FcεRI α chain results in upregulation of FcγRIII-dependent mast cell degranulation and anaphylaxis. Evidence of competition between FcεRI and FcγRIII for limiting amounts of FcR β and γ chains. J Clin Invest 1997; 99:915-25.

Finkelman F D, Katona I M, Urban J F, Jr., Holmes J, Ohara J, Tung A S, et al. I L-4 is required to generate and sustain in vivo IgE responses. J Immunol 1988; 141:2335-41.

Finkelman F D, Morris S C. Development of an assay to measure in vivo cytokine production in the mouse. Int Immunol 1999; 11:1811-8.

Finkelman F D, Morris S C, Orekhova T, Sehy D. The In Vivo Cytokine Capture Assay (IVCCA) for measurement of in vivo cytokine production in the mouse. Current Protocols in Immunology 2003:6.2.8.1-6.28.10.

Gallagher C M, Garri C, Cain E L, Ang K K-H, Wilson C G, Chen S, Hearn B R, Jaishankar P, Aranda-Diaz A, Arkin M R, Renslo A R, Walter P. Ceapins are a new class of unfolded protein response inhibitors, selectively targeting the ATF6α branch. eLife 2016; 5 e11878.

Li J, Wang Y, Tang L, de Villiers W J, Cohen D, Woodward J, et al. Dietary medium-chain triglycerides promote oral allergic sensitization and orally induced anaphylaxis to peanut protein in mice. J Allergy Clin Immunol 2013; 131:442-50.

Madden K B, Urban J F, Jr., Ziltener H J, Schrader J W, Finkelman F D, Katona I M. Antibodies to IL-3 and IL-4 suppress helminth-induced intestinal mastocytosis. J Immunol 1991; 147:1387-91.

Madden KB, Whitman L, Sullivan C, Gause W C, Urban J F, Jr., Katona I M, et al. Role of STAT6 and mast cells in IL-4- and IL-13-induced alterations in murine intestinal epithelial cell function. J Immunol 2002; 169:4417-22.

Morafo V, Srivastava K, Huang C K, Kleiner G, Lee S Y, Sampson H A, et al. Genetic susceptibility to food allergy is linked to differential TH2-TH1 responses in C3H/HeJ and BALB/c mice. J Allergy Clin Immunol 2003; 111:1122-8.

Osterfeld H, Ahrens R, Strait R, Finkelman F D, Renauld J C, Hogan S P. Differential roles for the IL-9/IL-9 receptor alpha-chain pathway in systemic and oral antigen-induced anaphylaxis. J Allergy Clin Immunol 2010; 125:469-76 e2.

Paul W E, Zhu J. How are T_{H}2-type immune responses initiated and amplified? Nat Rev Immunol2010; 10:225-35.

Saenz S A, Taylor B C, Artis D. Welcome to the neighborhood: epithelial cell-derived cytokines license innate and adaptive immune responses at mucosal sites. Immunol Rev 2008; 226:172-90.

Sanderson C J. Interleukin-5: an eosinophil growth and activation factor. Dev Biol Stand 1988; 69:23-9.

Sicherer S H, Sampson H A. Food allergy. J Allergy Clin Immunol 2010a; 125:S116-25.

Sicherer S H. Epidemiology of food allergy. J Allergy Clin Immunol 2010b; 127:594-602.

Sicherer S H, Wood R A, Stablein D, Burks A W, Liu A H, Jones S M, et al. Immunologic features of infants with milk or egg allergy enrolled in an observational study (Consortium of Food Allergy Research) of food allergy. J Allergy Clin Immunol 2010c; 125:1077-83 e8.

Strait R T, Morris S C, Yang M, Qu X W, Finkelman F D. Pathways of anaphylaxis in the mouse. J Allergy Clin Immunol 2002; 109:658-68.

Tabata Y, Takano K, Ito T, Iinuma M, Yoshimoto T, Miura H, Kitao Y, Ogawa S, Hori O. Vaticanol B, a resveratrol tetramer, regulates endoplasmic reticulum stress and inflammation. Am J Physiol 2007; 293:C411-18.

Tomasio S M, Harding H P, Ron D, Cross B C S, Bond P J. Selective inhibition of the unfolded protein response: targeting catalytic sites for Schiff base modification. Mol BioSyst 2013; 9:2408-16.

Zhao A, McDermott J, Urban J F, Jr., Gause W, Madden K B, Yeung K A, et al. Dependence of IL-4, IL-13, and nematode-induced alterations in murine small intestinal smooth muscle contractility on Stat6 and enteric nerves. J Immunol 2003; 171:948-54.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance. The present invention is further described by the following claims. The description in each section of this disclosure is intended to be read in conjunction with the other sections. Furthermore, the various embodiments described in each section of this disclosure can be combined in various different ways, and all such combinations are intended to fall within the scope of the present invention.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method of suppressing an allergic reaction to a food, the method comprising administering to a subject with a food allergy a pharmaceutical composition comprising an inhibitor of unfolded protein response (UPR).

2. The method of claim 1, wherein the allergic reaction is anaphylaxis.

3. The method of claim 1, wherein the food is selected from the group consisting of eggs, milk, peanuts, tree nuts, and fish.

4. The method of claim 1, wherein the inhibitor of UPR is metformin.

5. The method of claim 1, wherein the inhibitor of UPR is 4-phenylbutyrate (4-PBA).

6. The method of claim 1, wherein the inhibitor of UPR is a bile acid or pharmaceutically acceptable salt thereof.

7. The method of claim 6, wherein the bile acid is ursodeoxycholic acid (UDCA) or tauroursodeoxycholic acid (TUDCA).

8. The method of claim 4, further comprising administering a pharmaceutical composition selected from a composition comprising UDCA, a composition comprising TUDCA, and combinations thereof.

9. A method of reducing the likelihood of a food allergy, the method comprising administering to a subject susceptible to developing a food allergy a pharmaceutical composition comprising an inhibitor of unfolded protein response (UPR).

10. The method of claim 9, wherein the food is selected from one or more of eggs, milk, peanuts, tree nuts, and fish.

11. The method of claim 9, wherein the inhibitor of UPR is metformin.

12. The method of claim 9, wherein the inhibitor of UPR is 4-phenylbutyrate (4-PBA).

13. The method of claim 10, wherein the inhibitor of UPR is a bile acid or pharmaceutically acceptable salt thereof.

14. The method of claim 13, wherein the bile acid is selected from ursodeoxycholic acid (UDCA), tauroursodeoxycholic acid (TUDCA), and a combination thereof.

15. The method of claim 11, further comprising administering a pharmaceutical composition selected from a composition comprising UDCA, a composition comprising TUDCA, and combinations thereof.

16. The method of claim 1, further comprising administering a food antigen.

17. A pharmaceutical composition for use in treating or preventing a food allergy, the pharmaceutical composition comprising an inhibitor of unfolded protein response (UPR), wherein the inhibitor of UPR is selected from metformin, 4-phenylbutyrate (4-PBA), a bile acid or combinations thereof, preferably wherein said bile acid is ursodeoxycholic acid (UDCA) and/or tauroursodeoxycholic acid (TUDCA).

18. (canceled)
